(19) United States
(12) Patent Application Publication
(10) Pub. No.: US 2019/0321601 A1
(43) Pub. Date: Oct. 24, 2019

(54) Electromagnetic Motion and Tracking Seldinger Technique Access System: Introducing the EMMT STA System

(71) Applicant: Jaywant P. Parmar, San Luis Obispo, CA (US)

(72) Inventor: Jaywant P. Parmar, San Luis Obispo, CA (US)

(21) Appl. No.: 16/372,394

(22) Filed: Apr. 1, 2019

Related U.S. Application Data
(60) Provisional application No. 62/651,061, filed on Mar. 30, 2018.

(51) Int. Cl. A61M 25/09; A61M 25/01; A61M 25/02; A61M 39/08; A61M 39/10; A61M 29/00

(52) U.S. Cl. CPC A61M 25/09; A61M 25/0127; A61M 25/02; A61M 2025/0293; A61M 39/1011; A61M 29/00; A61M 2039/087; A61M 39/08

(57) ABSTRACT

The invention provides a variety of methods and apparatus for insertion of a needle and/or guidewire from a sterile tube. In some embodiments, a bungee cord holds the apparatus. The invention also provides methods of decoupling the needle and/or guidewire from the external magnets during an insertion process.

### RELATED APPLICATIONS

This application claims priority to U.S. Provisional Patent Application Ser. No. 62/651,061, filed Mar. 30, 2018.

### INTRODUCTION

There are 3 essential tools required for Seldinger Technique of Vascular Access: a needle, a guidewire and a catheter tube. Typically, these coaxially and laminated elements are sequentially assembled by hand at the time of procedure by the operator. A system is outlined in which the three essential tools are assembled coaxially in a factory and sterilized, being incorporated into a sterile enclosed magnetically active single use cartridge. At the time of use, the cartridge is mounted to a magnetically operated motion control handle which is suspended by its support stand for use, positioning the entire device and its 3 essential components ready for use by the operator's hand over the sterile operative field.

The EMMT STA System is comprised of (A) a multi-use motion control handle (MCH) and its supporting stand system (SSS), and, (B) a single use cartridge (SUC). The MCH actuators are coupled magnetically to elements within the single use cartridge (SUC) and described in detail in two variations. The nesting, placement and operating range of the MCH actuators, which can induce longitudinal and/or rotational motions of coaxially layered elements within the SUC, are critical for functionality. Iterations of these 2 primary variations that will be provided can allow increased or alternative functionality. The fundamental medical care procedure of “Seldinger technique (vascular) access” (STA), including modifications of this fundamental technique applied in extension, is the primary function of the EMMT STAS although the technology can be extended into other applications such as percutaneous drainage tube placement, or, PICC placement (US 20160096003, incorporated herein by reference as if reproduced in full below). Above all, the adoption of the EMMT STA system into medical practice will increase single healthcare provider confidence and efficiency, improve patient experience and outcomes, decrease contaminated waste and improve operator/environmental safety while decreasing overall cost.

### SUMMARY OF THE INVENTION

Inventive Concepts include any of the devices or methods described herein. Some aspects of the invention include:

A catheter or guidewire placement apparatus, comprising: a sterile enclosed tube comprising: a needle, a guidewire, and a first ferromagnetic component coupled to the guidewire, and, optionally, a catheter; one or more external magnets that are mounted to the exterior of the enclosed tube and coupled to one or more ferromagnetic components within the sterile tube; and wherein the largest dimension of the enclosed tube is the length direction and wherein the one or more external magnets comprises: a first external magnet that is coupled to the first ferromagnetic component that is coupled to the guidewire and wherein the first external magnet is translatable in the direction of the length of the enclosed tube; and a decoupling mechanism that allows the guidewire or needle to uncouple from the one or more external magnets.

The tube may be open at both ends or, in some preferred embodiments, open at one end. Preferably, the tube comes as a cartridge with all the interior components in sterile condition. The cartridge may then be opened at one end. The decoupling mechanism may allow the guidewire or needle to reversibly or irreversibly uncouple from the one or more external magnets. In some preferred embodiments, the decoupling mechanism creates a change in position of a magnetic actuator such that the central orifice becomes closed and impinges, crimps and locks upon the guidewire.

The invention also provides a catheter or guidewire placement apparatus, comprising: a sterile enclosed tube comprising: a needle, a guidewire, and a first ferromagnetic component coupled to the guidewire, and, optionally, a catheter; one or more external magnets that are mounted to the exterior of the enclosed tube and coupled to one or more ferromagnetic components within the sterile tube; and wherein the largest dimension of the enclosed tube is the length direction and wherein the one or more external magnets comprises: a first external magnet that is coupled to the first ferromagnetic component that is coupled to the guidewire and wherein the first external magnet is translatable in the direction of the length of the enclosed tube; and wherein the sterile enclosed tube is suspended from an overhead support by a bungee cord.

The invention also provides a catheter or guidewire placement apparatus, comprising: a sterile enclosed tube comprising: a needle, a guidewire, a dilator, a sheath, and, optionally, a catheter; the guidewire disposed within the needle; wherein the sheath and dilator are cylindrical; the dilator is slidable over the guidewire and the sheath slidable over the dilator; and preferably where the sterile enclosed tube is suspended from an overhead support and preferably suspended from the support by a bungee cord. This is demonstrated in FIG. 3 where 3 and 4 (dilator and sheath) are placed external to 5 (motion control handle). This way they can be hand operated, one hand holding the motion control handle (5) and the other hand pushing the sheath and dilator forward. This hand operated system is preferably designed with some flexibility be built into the needle. That way, the exposed needle can be advanced a short distance over the guiding guidewire safely, allowing the dilator to have a “gapless” transition over the vessel wall. If the needle has to be pulled back, the dilator would advance over the guidewire and would encounter more difficulty in parting the tissues due to the gap between the guidewire and the dilator where the needle must be interposed. This sort of catheter/needle advancement is very easy to do and would preferably include shortening the bevel of the needle so that flex at the needle guidewire transition would minimize the exposed point of the needle tip. Alternatively or in combination, a bevel could be rotated from bevel up to bevel down position, protecting the barb tip against the concavity of the guidewire shaft.

The invention may also includes apparatus with the needle and needle insertion (optionally including the guidewire and catheter). In some aspects, the invention comprises the sleeve in conjunction with any of the apparatus described herein. In any of the methods or apparatus, the external actuators can be operated by motor or by hand.

The invention includes methods of using any of the apparatus described herein. The invention is not limited to the specific concepts discussed above and includes any combination or subcombination of the elements in the apparatus and any combination or subcombination of steps that are described herein. Various concepts of the invention have been described in conjunction with the word “comprising” which means including; in any of the concepts, “comprising” can be replaced with “consisting” or “consisting essentially of” to describe narrower versions of the invention that exclude additional components or substantially exclude additional components.

Various details and additional aspects of the invention can be understood with reference to the figures as discussed below.

### BRIEF DESCRIPTION OF THE DRAWINGS

FIG. 1A illustrates a system including a supporting stand and catheter or guidewire placement apparatus.

FIG. 1B illustrates an EMMT STA cartridge system

FIG. 2 is a schematic illustration of a needle guidewire EMMT system cartridge with guidewire releasability.

FIGS. 3A and 3B illustrate operation of a coupling of the magnetic actuator of the guidewire (or needle) may be releasable via magnetic field shape change of the external actuator.

FIG. 4 illustrates a needle, guidewire, sheath EMMT system.

FIG. 5 is a cross-sectional view of a needle, guidewire, sheath EMMT system.

FIG. 6 is a drawing illustrating the integration of the sheath and handgrasp in the device shown in FIG. 7.

FIG. 7 is a photograph of the sheath and handgrasp of a device of the type described herein.

Motion Control Handle (MCH) and supporting stand system (SSS)

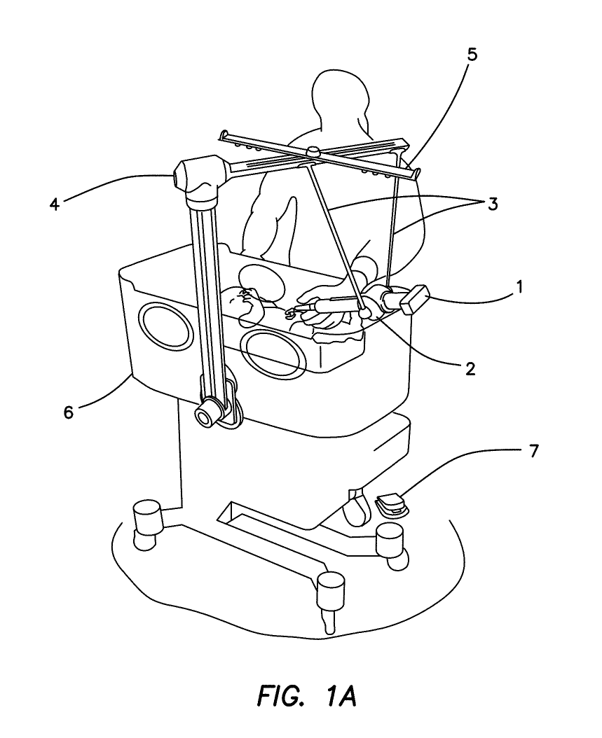

Please refer to FIG. 1A.

The typically lightweight (preferably 4 kg or less, typically 200 g-4000 g) MCH has magnetized motion control actuators integrated within it. The MCH and the internal actuators are guided by the operator's hand. The MCH typically has an elongated shape which may be coiled or spooled, and, it may be flexible or rigid, and, it may be used with a variety of supporting stand systems (SSS). For example, it may be hung balanced from an IV pole by a supporting bungee which is connected to a rotational ring bearing holding the MCH. The term “bungee” has the conventional meaning of an elastic cord, composed of one or more elastic strands usually covered in a sheath.

The MCH is then grasped by a handle and guided and rotated by the operator hand. Grasping the handle translates to grasping with motion control system assistance the sterile tools required for STA (Seldinger Technique Access) that are held within a single use cartridge (SUC). The net result of the design is that the whole device becomes very slightly weighted in the operators hand thereby optimizing the tactile sensitivity of the human operator's hand to the forces which are encountered by the nested internal tools required for STA as they are manipulated with motion control system assistance. Indeed, the internal components within the SUC may become decoupled and recoupled from the MCH actuators and MCH handle by traction provided by the operator's hands between the MCH and the exposed and grasped internal component. The MCH allows the operator to control its motorized actuators with joysticks, touchpad sensors, footswitches, voice control, and, other types of computerized and optionally haptic enabled operator user interfaces, thereby assisting the operator to use the sterile tools within the cartridge to achieve STA with extreme efficiency. As the device brings “haptic” machine feedback to the operator, there is also a possibility for the MCH to provide a direct network connection to elements of the electronic medical record (EMR), the GPS network, cellular networks and other digital networks. The bungee 300 is preferably hung from 2 points on an overhead support 4 that may be rotatable and may be attached to a stand or a neonatal bed 600.

Single Use Cartridge (SUC)

Please refer to FIG. 1B—System EMMT STA Cartridge Overview

Conceptually, the cartridge is an elongate enclosure typically of flexible plastic which is placed within or otherwise operably connected to the MCH. The cartridge contains some or all of the basic disposable components required to perform Seldinger vascular access, typically a needle, guidewire, and, catheter/sheath+dilator. The cartridge serves as a protective sterile transport vessel and as a protective barrier for the internally nested elements, and, it may have an integrated sterile sleeve MCH grasp cover. It may also incorporate packaging related subcomponents that while providing a sterile barrier and protective needle covering also may also allow for locking the motion (“screw-caps”) of the internal components so as to aid in the mounting/dismounting of the single use cartridge to the motion control handle before use. Different cartridge internal element ensembles will be considered. Each ensemble contains two essential components: a guidewire 1, typically a composite flexible wire transitioning in stiffness from floppy at the proximal tip 21 to a rigid shaft at the distal end, and, a hypodermic, thin-walled, beveled or flat-ended needle 2, typically made of stainless steel or nitinol alloy. The hypodermic needle may have outer etching to increase sonographic echogenicity, and, it could be made of alloy such as Nitinol in order to support flexibility or even temperature related memory positions. The guidewire is typically a composite elongate wire structure of overlapping segments joined lengthwise in order to transition smoothly in stiffness, from very soft shaft wire with a rounded and flexible/floppy tip (aka—atraumatic), to very rigid and stiff shaft on the distal (aft) end. This may alternately be described in terms of stiffness, with the material within 1 cm of the proximal end having a stiffness that is at least 20% or at least 50% less stiff than the material within 10 cm of the distal end. The fabrication of guidewires involves significant engineering and has achieved a very sophisticated level. Through the cutting tip of the hollow needle, the guidewire is further advanced from pressure on its aft end “non-traumatically or atraumatically” within the vessel lumen leading with the soft and floppy guidewire tip and the sides of the distal guidewire gently siding along the endothelial lining as it passes through the bloodstream and is advanced within the vascular tree. For modern manual “hand operating” Seldinger access within compact operational spaces, a broad range of shaft stiffness is required of a short guidewire, typically less than 70 cm and usually 30 cm. After pushing the free needle through the wall of the vessel by hand, usually under direct ultrasound visualization, blood returns and upon seeing this blood return from the back of the needle unimpeded, then, the operator confirms that the needle tip is in good contact with the central lumen, and, then, she feeds the guidewire, floppy tip first, through the needle. The moment the guidewire floppy tip becomes exposed from the needle, it will encounter varying resistance depending on needle tip position, sensed by the pushing operator hand on the aft of the guidewire. This sort of tactile sensitivity is needed because improper probing can destroy of vascular tissues, for example if the needle tip is centered in the mid vessel wall tissues and not in the blood pool within the central lumen then guidewire advancement may actually penetrate into the vessel wall and cause dreaded vessel-wall “dissection.” Thus, tactile sensitivity is a further confirmatory sign of good needle tip positioning. The MCH supporting stand system (SSS), possibly incorporating a bungee or elastic linkage, is designed to provide weightless, low inertial/mass, support to the MCH so as to easily present the forces encountered by the advancing guidewire tip within the vascular conduit, to the operator through his/her supporting hand through the MCH. This is a primary design feature of the system.

Through description of the EMMT STA system using two different cartridge configuration embodiments, the advantages of the EMMT STA system over traditional hand operation STA will become apparent, and, underpinning design features of this system enabling a successful automation will be briefly explicated.

EMMT STA System Cartridge Ensemble #1

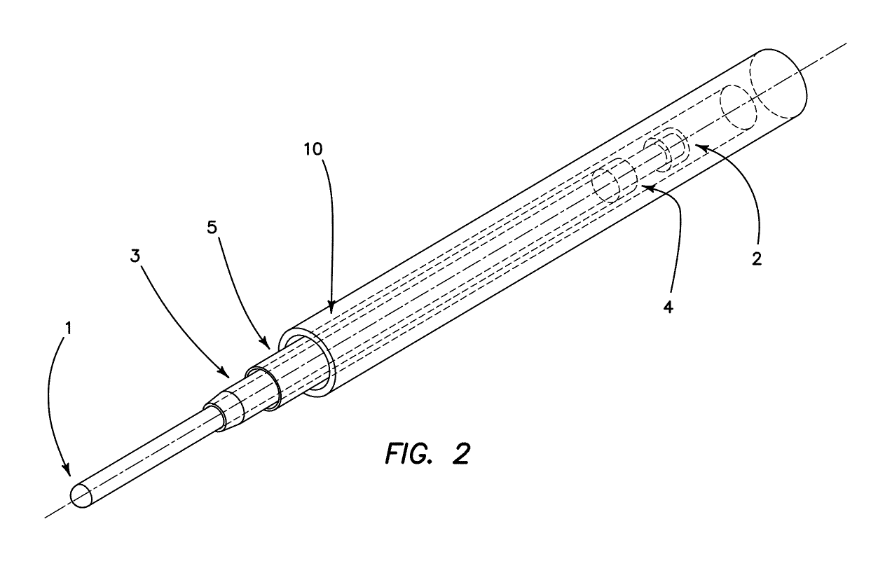

Cartridge ensemble #1 is comprised of, from internal outward (1) a guidewire and (2) a needle. Magnetic actuators are physically connected to the guidewire and needle within the cartridge at their distal ends. The magnetized actuators cannot pass over one another or overlap as that condition will usually cause both mechanical and magnetic interference. The magnetic actuator coupling 6 of the needle is fore, the guidewire aft 5. Furthermore, the coupling of the magnetic actuator of the guidewire (or needle) may be releasable via magnetic field shape change of the external actuator 14, 13 inducing an ungrasping of the internal cartridge actuator such that the guidewire is released from it (FIG. 3A, 3B). An iteration of cartridge #1 (FIG. 1B top) is removing the needle (2), needle actuator (6), and guidewire (1), leaving only a releasable guidewire actuator (5). This would allow an “empty” EMMT cartridge (10) containing an “empty” guidewire magnetic actuator (5) to grasp a blood contaminated guidewire, possibly from the free end protruding directly from within a patient's body, and, pull it into a cartridge chamber for rapid and efficient environmental securement and disposal. Alternatively, the guidewire of ensemble 1 (FIG. 1B top)) could be advanced and then selectively released if the operator so desired, providing a very realistic “bail out” procedure of over the guidewire catheter exchange moving from one EMMT cartridge system to another, or, by converting to traditional hand operated catheter guidewire technique. A variety of release mechanisms other than magnetic field shape change are possible, which could function via non-contact-requiring energetic transmission through the cartridge such as by sound, radiation or electromagnetic fields through the catheter housing enclosure, or, manipulation or separation of the MCH mounted external driving magnets. However, (1) external rotational motions of static magnets relative to the longitudinal axis of the cartridge housing or (2) changes from dipole to quadrapole field shapes may represent the most straightforward designs as simple external ring magnets and simple manipulations of these are the only requirements.

In some preferred embodiments of any of the apparatus, a “notched” guidewire that allows mechanical locking of the internal magnet with matching “notch” to the guidewire when the external magnet is rotated in the longitudinal axis induces “binding” of the internal components. The binding force in this case is the result of the rotational force of a ring magnet against the fixed (nonrotating) longitudinal axis of the guidewire shaft. The components may be “unbound” when the external actuator is rotated back so as to align with the long axis of the guidewire shaft.

EMMT STA System Cartridge ensemble #2 includes from internal outward and aft-fore:

(1) A guidewire

(2) A needle

(3) A tapered dilator

(4) A vascular introducer sheath tube (or drainage catheter).

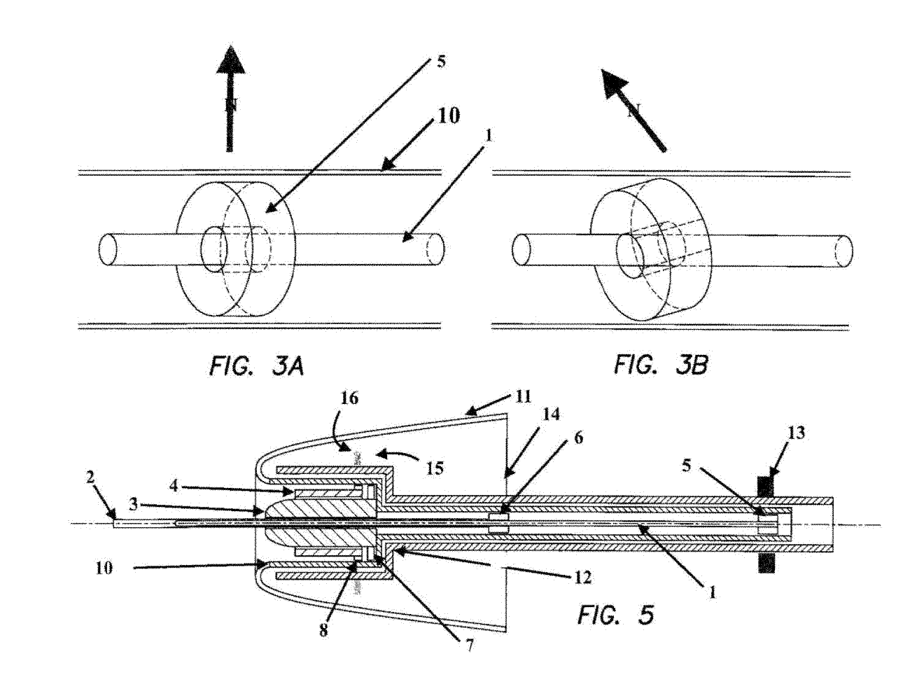

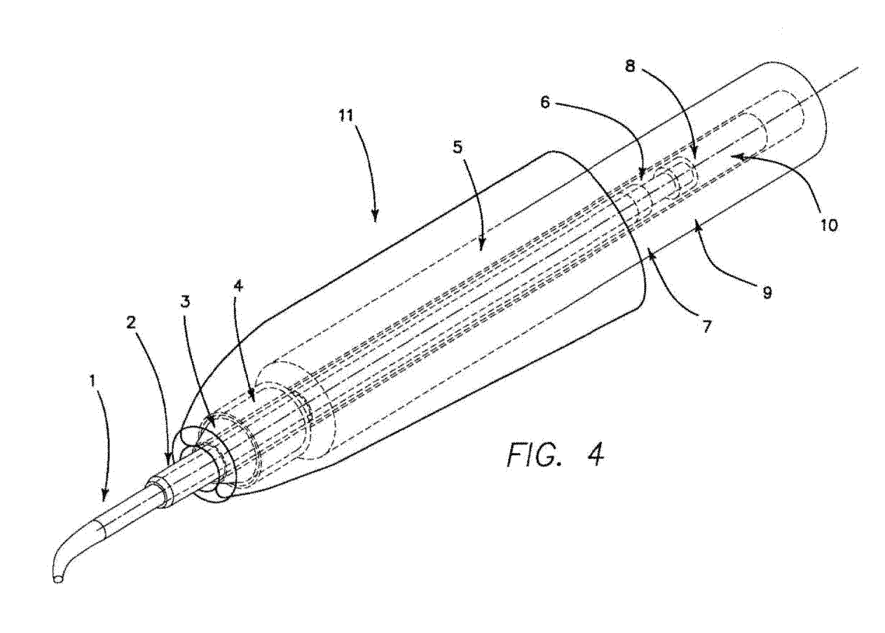

Cartridge ensemble #2 is presented in two forms: FIG. 4 is partially hand operated, and, FIG. 5 is full motion control.

The actuators cannot cross over one another or overlap as that condition will cause both mechanical and magnetic interference, thus this is a primary design factor and limitation. The guidewire is central and it has the needle surrounding it. Over the needle, there is a tapered dilator which has mounted over it a vascular introducer sheath tube or drainage catheter. This sheath or drainage catheter implant can be advanced over the needle and guidewire when the internal dilator is in place, or, over the guidewire and dilator alone after the needle has been retracted. The sheath (or drainage catheter) 4 and its internal dilator comprise an implant which is eventually separated from the cartridge and motion control handle and then left with the patient as a temporary implant. When the internal dilator 3 is removed, the sheath may serve as an implant tube conduit from the ambient environment directly into the blood-filled vein, also known as a “venous access site.” Similarly, a modification of Seldinger access occurs when the guidewire is placed into an abscess cavity using a needle under imaging guidance. Thus, a drainage catheter could be positioned within an abscess by a modification of classical Seldinger technique using the system. The EMMT STA System needle and guidewire are constrained immediately after their use and contamination by blood or abscess fluid. In the modern hand operated technique, the needle and guidewire must be handled according to “contaminated sharps medical waste” safety policies at all times, both within the sterile field during the procedure, and, according to local waste management polices after the implantation procedure is complete. In the state of California, contaminated sharps medical waste is required to be stored after use in hard shell containers that are sealed, and, then incinerated for final disposal. Contaminated needles and guidewires in a traditional sterile are cumbersome and require careful attention in order to minimize risks during use and during placement into hard shell containers. Contaminated guidewires in particular can be quite difficult to constrain and may “spring” into unconstrained state, flinging fluid droplets onto environmental surfaces after use. Typically, guidewires require a large working space for use, typically a separate “back table” sterile working space that is required to lay out the used guidewire and re-constrain it into its packaging tubular constrainment spool which is the best technique. Often times, constrainment spools are accidentally discarded or lost in the operative field. In this case, contaminated guidewires are often handled in a variety of manners and many of these present significant risks to operators and the environment as the guidewires are slippery and fluid contaminated, and, they may “spring” back to their unconstrained state if they are improperly constrained without the constrainment spool.

The EMMT STA system needle and guidewire are advanced out and then fully retracted back into the protective cartridge housing, ready on demand to be used again or to be disposed. There is no operator concern for disposal, less space is needed, and, contamination risks are mitigated. During final disposal of the EMMT STA contaminated needle and guidewire, the outer cartridge shell provides a protective barrier which is carried to the hardened contaminated sharps medical waste container. Thus the concept of the bedside “sterile operative working space comprised of a (1) sterile field and (2) “back table” is significantly improved in terms of operator ease, maximum sterile barrier technique, operator and environmental safety and at the same time minimizing the physical space needed to accommodate the procedure. In fact, in many cases, the EMMT STAS would obviate the need for a human sterile scrub assistant and the additional back table sterile field space beyond the access site sterile field which is often required in bedside procedures. Indeed, the passing of contaminated needles and guidewires from a “back table” and into and out of the center of the surgical field would be completely obviated. This is a significant advancement when we consider the location of many of these “bedside procedures” which are becoming more and more common in hospital practice, particularly in interventional radiology (IR) practice. In these bedside procedures, the sterile operative field is right on the patients skin and includes a drape laying on the patient and their bed surrounding the sterilized skin access site onto which all sterile tools are laid out. The patient may be lying in a hospital bed, or, on a hospital gurney, or, on a CT scanner table, or, on an ultrasound procedure table. Thus a supplementary “back table” is often required, and, moving contaminated sharps between these 2 sterile areas presents a significant set of potential risks that would be completely mitigated.

The EMMT STA system guidewire actuator is the furthest aft, followed by the needle actuator, then by the dilator actuator and then the sheath actuator. The sheath and dilator could also very well be hand operated and advanced manually after separation from the MCH through contact and counter tension against the MCH which would be linked to the internal guidewire. This is likely to be an efficient and cost effective design, and, it is further explicated in FIG. 4. But, in its most automated form, the EMMT STA system will have the entire access ensemble, including the (a) guidewire, (b) needle, (c) dilator and (d) sheath each independently controlled through the cartridge housing via its own dedicated magnetic actuator. This is be explicated in FIG. 5, and, this system, although more mechanically complex, importantly allows a “virtualized” locking of the needle layer to the dilator layer through computer control, a significant feature that may be very desirable toward successful clinical operations.

Through force/position sensor integration between the magnetic coupling, forces at play on the tissue contacting elements of the EMMT STA system (needle, guidewire, dilator, sheath) may be transduced by the internal actuators in order to improve functionality. One can imagine the typical scenario where the operator will be using the EMMT STA system to enter an arterial lumen. The operator will first select to expose the needle. The needle tip will be then be pushed by hand via pressure on the MCH or needle and advanced bevel up through the dermis, subcutaneous tissue, outer vessel wall, inner vessel wall, and, finally into the vessel central lumen. During this process of penetration of the needle, the guidewire tip will be positioned approximately 1-2 cm behind the needle tip. The operator will ultimately force the needle tip through the vessel wall and into contact with the flowing blood within the vessel lumen. When the needle tip is position within the central artery or vein lumen, the blood pressure will push blood back through the needle lumen. The force of the blood upon the internal guidewire may be transduced based on position and orientation of the attached (driven) magnetic element, and, its displacement from the driving magnet isocenter. For example, in the case of an artery, the arterial blood pressure will cause pulsatile motion of the guidewire translated to the driven magnetic element which is displaced from the driving magnet isocenter. This displacement will translate to a compensatory force against the motion control handle and will eventually translate to the operator's hand. The forces at play will be visual, tactile, and visually and/or computer haptically observed by the operator through interaction with the EMMT STAS to allow her to well position the first needle prior to guidewire advancement into the lumen. In an alternative example, in the case of venous lumen puncture, the forces of augmentation or right atrial activity could be sensed, but, these would be 2 orders of magnitude scale difference. Other sensors could be integrated into the needle to sense contact with blood. Alternatively, the guidewire could be retracted allowing blood to be visually observed and vented into a sight chamber, and/or a suction force could be applied to fluid within the cartridge bringing blood into a site chamber visible to the operator. Ultimately, when the guidewire is advanced, if the needle is well centered in the lumen, the guidewire will initially encounter almost no force of resistance to advancement for its first few centimeters. Force of initial contact will be very light and based on the floppy guidewire tip. The guidewire will eventually self-center with either a straight or bowed back tip in the lumen as it is advanced. Force of resistance will gently build as greater lengths of guidewire are advanced to contact, slide and brace against the sometimes tortuous vessel lumen. The goal of guidewire advancement is to build stiffness gradually from the atraumatic floppy tip in the front to eventually match and possibly surpass the needle stiffness in the back so that the monorail will support a very stiff dilator to pass over the guidewire without kinking and buckling it.

Operator Experience:

Improving the operator experience is required for success of the device. After working with the EMMT Clean PICC system, I have come to the conclusion that an automated Seldinger access system is feasible and will offer significant advantages over the current manual approach. Much of the success of this design relies on weight, balance and inertia of the EMMT system. In particular, all of these factors can be very well balanced to allow procedural sensitivity using a bungee or bungee-like suspension connection (ultimately) to the ground via a stand. If the weight of the device is well balanced, the device can essentially become near weightless in the operator's hand, allowing the operator to sense and respond to the tactile cues important to successful interventional radiology procedural performance. In addition, these tactile cues can become augmented via haptics and motion control through the added functionality of the device. In the case of Seldinger access, a number of individual elements are assembled and associated in free space relative to the handle, and, the elements are individually and collectively grasped and controlled by the operator. The EMMT modification of STA nests the basic components necessary into a device that mechanically automates these steps in a hand held tool while preserving the tactile human operator sensitivity to the components in a device that can “float” and move almost weightlessly with a patient's access site, as patients often move unexpectedly during a bedside procedure. Thus, due to the bungee-like support, preferably, the device moves moves opposite gravity at a rate that is at least double that which would be obtained by the unsupported device.

Many types of supporting stand systems (SSS) could be designed. In preferred embodiments, a bungee-like suspension connection has one or any combination of the following properties: 100% elongation for a weight in the range of 1-100 kg, 2-100 kg, or 5-50 kg; or a spring constant in the range of 1 to 150 N/m; 2 to 100 N/m, or 5 to 70 N/m. Of course, the connection should be strong enough not to break during operation. A key feature of the bungee is that it allows a high degree of mobility in cases of unexpected motion, adding to the safety of the device. The bungee also allows extreme operator tactile sensitivity to exposed needle, catheter, guidewire. It is easy and intuitive for the operator to guide the device, adjust to the weight of the device in the hand and then explore various hand grasp positions all while sensing what the exposed elements are encountering through hand and possible haptic enabled operator inputs to the MCH. A more mechanical linkage (versus a bungee) will not be as safe and will not provide this “weightless” operator experience. There are some features of the bungee that we are finding. The longer the bungee, the better, because it allows more motion and sensitivity, as the pendulum is long; preferably, the bungee or bungee-like connection has a length of at least 50 cm or at least 1 m. The bungee and/or the system handle can be constrained by guides in order to limit motion in certain regions or hold the handle in a “stand-by” position, adding to ease and flexibility in procedural set up. A variation of the bungee suspension (for enhanced stability) is illustrated in FIG. 1 in which two bungee-like cords 300 are connected to an overhead support and connected to opposing lateral sides of the MCH, preferably via a ring bearing 200.

As an example, in one embodiment, the device MCH and cartridge assembly weighs 2200 grams and is 100 cm in length, with the center of gravity located 60 cm aft of the front mounted operator handle. The bungee attachment is located slightly aft of the center of gravity (cg) so that the handle is weighted slightly in the operator's hand. The handle weight is very light and adjustable: approximately 10-100 g seems to be an optimal range depending on the size of the operator. The bungee stretches from its nominal length, 20 cm, to its weighted length, 40 cm, with the full weight of the assembly. The bungee hangs from a rolling IV pole moved in position so as to suspend the assembly in a position that is natural for vascular access to be performed by the operator who may be standing or seated beside the patient. The MCH will hang at typically a 30-45 degree angle of incidence to the skin. A parking hook may be positioned adjacent to the elongate barrel of the MCH chassis allowing the chassis to be positioned on “stand-by” for operator use. The floor mount could have locking/unlocking wheels or supports operated by foot switch, allowing an operator to control the assembly position/orientation with a foot switch/pedal. It is possible that one stand may hold several assemblies in close proximity and positioned to enable rapid interchangeability of these assemblies to the operator in one procedure for multiple primary STAs in one procedure, or, other secondary EMMT assisted procedural instrument operations following STA, and, through that STA.

A contemplated example highlighting the operator experience is as follows. The MCH is mounted with a cartridge by the operator and then is placed in “stand-by” position. The patient skin access site is prepared and draped to be sterile. When the operator is ready to proceed with the sterile procedure, the MCH handle is grasped by the sterile gloved operator hand, and, the MCH is given a command to expose the sterile needle. The needle, and/or MCH, is grasped by the human operator's sterile gloved hand and this guiding contact is used to advance the needle tip into the artery or vein, typically under sterile ultrasound probe image guidance by the operator and with secondary assistance of digital fluoroscopy. With fine changes in angulation and advancement under operator control, the needle tip is eventually positioned within the vascular lumen and in open contact with the blood pool. The EMMT system is able to transduce arterial or venous blood pressure/pressure signals via the lumen of the first needle and thereby is able to help the operator to identify good positioning of the needle tip within the lumen of the artery or vein. The operator then triggers and controls advancement of a guidewire through the needle. If the advancement is met with little resistance, the operator can perceive this based on mechanical feedback from the MCH handle, visual inspection of the guidewire at the front of the cartridge, and, possibly through computerized “haptic” feedback to the operator from the MCH. After the guidewire is advanced, the EMMT system allows the operator to (1) retract the needle over the guidewire, (2) rotate that needle and readvance it in conjunction with the dilator and sheath to position the sheath ready for advancement to access the artery or vein. The sheath and dilator could then be advanced over the guidewire and needle which will be held in a suitable position by the MCH and guiding operator hand.

### DESCRIPTION OF THE FIGURES

FIG. 1A: MCH+SSS Example

The figure demonstrates an EMMT STA device MCH (12) which is hung mounted in a rotating bearing (200) which is supported by 2 bungees (300) that are connected to a support stand (400) with integrated lighting posts (500) that is mounted to a wheeled neonatal patient bed (600). The MCH handgrasp hangs over the patient's body and is utilized in a sterile field. The support stand may alternatively be mounted to an IV pole with wheels. A footswitch (700) may be utilized to provide additional control to the MCH or elements of the stand (e.g., raise or lower the stand and/or adjust lighting). The operator grasps the suspended MCH by its hand grasp, and, interacting with the MCH using joysticks, touchpads, voice controls, footswitches and/or other means. Data from the MCH can be displayed to the operator in a variety of electronic and non-electronic means during a procedure.

FIG. 1B: EMMT STA CARTRIDGE OVERVIEW—Fundamental cartridge ensembles of the EMMT STA system cartridge basic design are outlined.

Ensemble 1 (top FIG. 1B) contains the most fundamental tools required for Seldinger access using an EMMT system with 2 magnetic actuators. The central guidewire (1) will have a floppy tip (left, 21) and will transition to a stiffer shaft distally (to the right). Typically a 0.018 in guidewire of mandril design would be used, but, other guidewire diameters and designs could be used. Mounted over the guidewire, a thin walled hypodermic needle (2) is positioned. The guidewire magnetic actuator (5) and needle magnetic actuator (6) allow independent motions including rotation and/or linear translation through coupling to the MCH. The entire ensemble can be contained in a cartridge housing (10) which may be plastic. The cartridge housing may have a plastic sleeve fused to its front end, which is not depicted in FIG. 1B. The guidewire (1) is released after passing from the skin into the vessel lumen, allowing manual work over this first guidewire in order to place the sheath by hand.

The ensemble shown at the bottom of FIG. 1B is more complicated and contains additional elements to place an introducer sheath conduit or drainage catheter with a significantly larger diameter than the first guidewire into the vessel through the skin, using an EMMT system, over the first initial placed guidewire (1) with 4 magnetic actuators. The additional components are a dilator (3) and its proximal magnetic actuator (7) as well as a sheath or drainage catheter (4) and its proximal magnetic actuator (8).

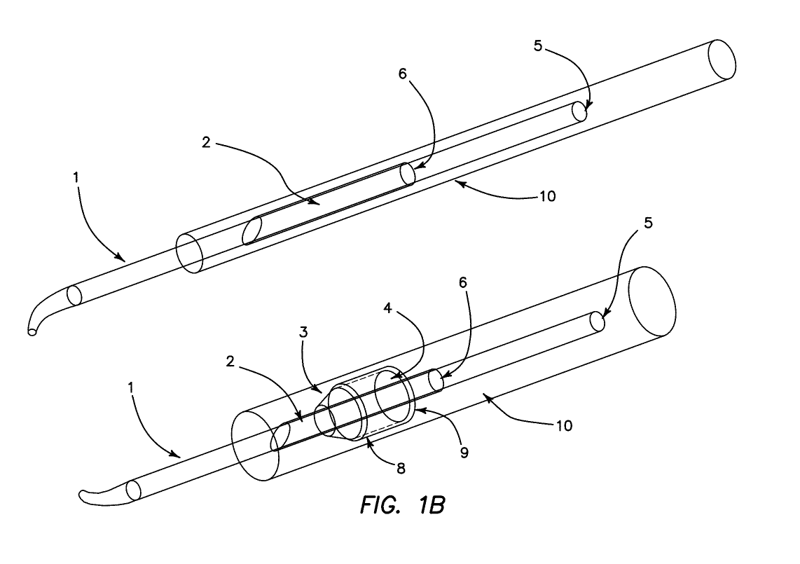

FIG. 2: Needle guidewire EMMT system cartridge with guidewire releasability

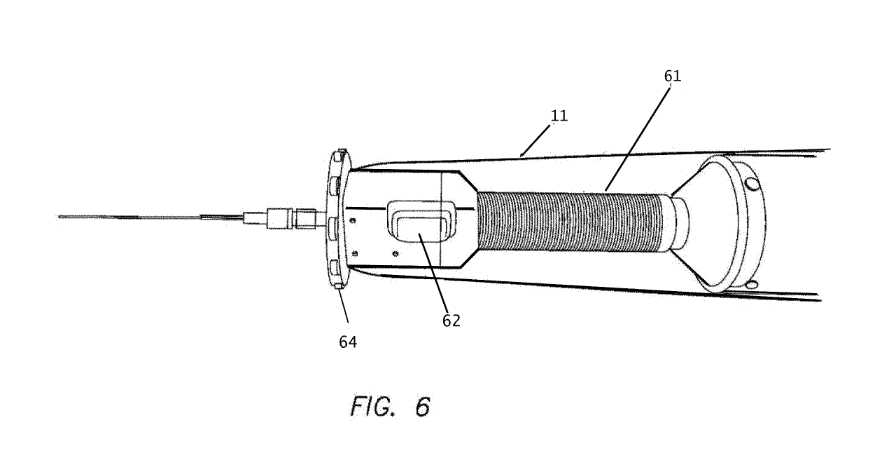

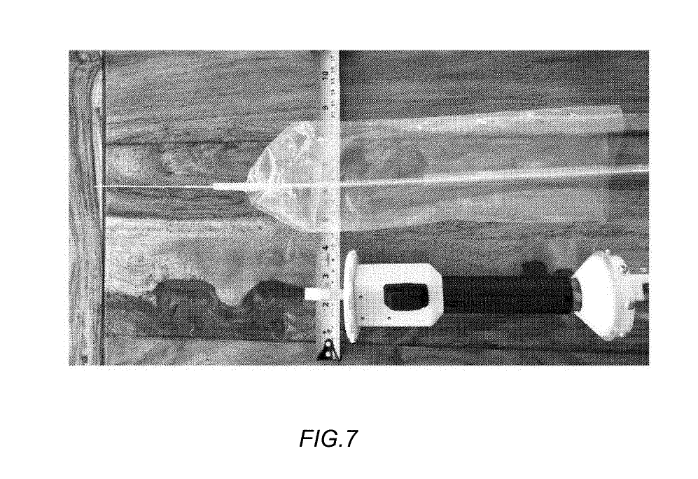

The central guidewire (1) with floppy atraumatic proximal tip has a magnetic actuator (5) at or toward its distal end which is releasable, for example, as is demonstrated in FIGS. 3A and 3B. The closely overfitting and encircling needle (2) is mounted over the guidewire and has a magnetic actuator at or toward its distal end (4). This needle may be beveled or may be flat round ended. These factors become important in the way that a dilator may be tapered to it so has to protected its cutting surface from a sharp encounter when this is not desired, as in the later stages of dilation during STA. A tube (sheath) (4) which may serve as a vascular introduction sheath or as a drainage catheter (with one endhole and possible multiple sideholes) is mounted over the needle and this may or may not include an internal, proximally tapered, internal space filling structure (dilator) (3) as detailed in FIGS. 3 and 4. As stated before, the dilator is a space filling structure and stiffener which allows the tissues to be parted by the tip of the catheter+dilator combination by force placed on the proximal shaft of the catheter+dilator, in order to create a larger diameter (typically) circular opening of the tissues surrounding the guidewire which was placed through the initial needle. The single use cartridge housing (10) is placed within the MCH during operation. A plastic sleeve (11) may be fused to the front end of the cartridge and used to cover a portion of the MCH, and the handgrasp, in order to form a sterile protective barrier for single use within the sterile operating field. The MCH handle is in this way isolated from the sterile field. FIG. 6 illustrates a handle (handgrasp) 61 with switch 62 that can be used to control motion of the guidewire and other optional, slidable elements. The optional sleeve 11 can be held between the handle and disc 64. More preferably, the sleeve 11 can be affixed to a cartridge for loading into the MCH (see FIG. 7) that would cover the front portion of the MCH. The invention includes the concept of a disposable cartridge comprising a sleeve that affixed near a proximal end of the cartridge and, in operation, the sleeve drapes over the front portion of the MCH.

FIG. 3: Guidewire releasability

The feature of guidewire releasability in the EMMT system opens up a possibility for operator “bail out” after placement of the first guidewire. If this guidewire can be released from its magnetic actuator, this would free the guidewire from the cartridge assembly and EMMT handle, allowing the standard manual procedure may continue unimpeded. It is certainly more elegant than a wire cutter, and, safer. Thus, guidewire releasability is important to the EMMT system in order to meet high level operator demands. A physician interventional radiologist (IR MD) operator will demand that the EMMT tools have a basic level of flexibility and integration to the traditional hand operated approach. One IR motto comes to mind, “never give up access!” Magnetic actuator releasability allows the EMMT system to become completely integrated with the traditional manual approach by never abandoning the often hard fought initial Seldinger guidewire access site.

By changing the shape of the external magnetic field, the actuators internal to the cartridge may be manipulated such that the guidewire and catheter may be released, and, possibly, re-grasped. The FIG. 3A demonstrates an uncoupled guidewire actuator (5). As the field is aligned perpendicular to the axis of the guidewire (1), the central orifice of the solid ring guidewire actuator is opened wide to allow the guidewire to pass unimpeded. FIG. 3B demonstrates a change in position of the magnetic actuator such that the central orifice becomes closed and impinges, crimps and locks upon the guidewire. The guidewire is held in a tubular constrainment housing, which is the cartridge housing (10), which constrains and determines the guidewire long axis, and, turning the diametrically aligned ring magnetic actuator diametric axis away from perpendicular to the guidewire axis and its tubular constrainment housing (less than 90 degrees, maybe 80 degrees or 75 degrees) effectively couples the motion of the actuator (5) to the guidewire (1) through mechanical binding/linkage of metal guidewire to metal magnet or a treated or shaped surface of the typically Neodymium magnet. Thus, magnetic field shape change can induce locking and unlocking of the actuator (5) to the guidewire (1). A similar process could apply to a catheter, with a ring or shaped magnet mount on or toward its distal end. The system could alternatively or in addition utilize geometric/mechanical coupling structures such as screws and hooks. Additional shaped lips and flanges could be arranged in order to facilitate locking for linear movement. The feature of magnetic actuator releasability will allow important additional functionality of any guidewire based EMMT system. Furthermore, magnetic field shape changes can induce or signal a variety of functional changes in internal elements of the cartridge, some of which could be quite complicated and internally powered beyond the power given through magnetic force coupling. For example, the magnetic field shape change could mechanically release or trigger stored energy within a compressed spring mechanism within the cartridge to release the stored kinetic energy of that spring. These sorts of mechanisms could be utilized to release stents or embolization coils, trigger electrical contact to complete a circuit, or, possibly to actuate valves to inflate and/or deflate angioplasty balloons.

Other decoupling mechanisms could include disengaging and removing the coupled magnets that are mounted on the outside of the external tube, or, less preferably, severing mechanisms that would cut the guidewire, even a hand operated wire cutter with grips on the external side of the tube and blades on the interior.

FIG. 4: Needle-Guidewire Dilator-Sheath EMMT system Consider an embodiment of the needle-guidewire EMMT STA system of FIG. 1B, and, then consider an embodiment of “manual advancement” of the dilator and sheath over the guidewire and needle while applying a backward countertension against the MCH handle and linked guidewire and needle which are designed to be contained in a cartridge housing immediately after use through EMMT control via the mechanically and magnetically linked MCH handle.

With reference to FIG. 4, the innermost layer is the guidewire (1), which is housed within a sterile single use cartridge (10) with an integrated flexible membrane sterile sleeve (11) that is mounted to serve as a sterile cover for the MCH (12). The sterile single use cartridge (10) may incorporate a distal sleeve component (11) that will cover the MCH (12) handgrasp. In some cases, when ready for use, the single use cartridge does not contain within its outer hard plastic tubular enclosure under EMMT control a portion of the dilator and the entire vascular introduction sheath/drainage catheter implant. The sheath/catheter (4) should be distinguished as the temporary patient implanted device component of the EMMT STA, which is designed for use by the patient for several hours to several days. The goal of the EMMT STA system is to place this sheath or catheter with extreme efficiency. The MCH (5,7,9) is a multi-use device. The cartridge is for single use, and, all the portions of the cartridge that are not the sheath or catheter become contaminated medical waste following the procedure. In this depiction, the single use cartridge sterile enclosure incorporates both a hard plastic tube portion (10) and a flexible fabric or plastic sleeve portion (11) that may initially enclose the implant on a portion of the dilator. This outer sleeve portion could eventually be folded back on itself to provide a sterile grasping cover (11) for the MCH hand grasp when it is actively utilized in sterile procedure.

The dilator (3) and sheath/catheter (4) are exposed in FIG. 4, as a sterile sleeve (11) is retracted over these previously sterile enclosed elements and folded back over the MCH. As the elements are exposed, the previously sterile inner surface of the enclosure is pulled back over the MCH thereby enveloping the handgrasp and now providing a sterile grasping surface of the MCH.

At the back end of the guidewire, a guidewire magnetic actuator element (5) is positioned (see FIG. 5). This may be releasable. The SUC elements (1, 2, 3, 4, 5, 6, 7, 8, 10, 11) and EMMT MCH (12,13,14) are designed to have a length great enough (in some preferred embodiments at least 50 cm or at least 1 m; and typically 2 m or less) to fully retract and enclose the needle (2) and guidewire (1) into the hardened tubular plastic enclosure (10) after use for environmental protection and safety.

The thin walled needle (2) is always positioned over the guidewire (1) and is slidable and (typically and preferably) rotatable over the guidewire. The inner diameter of the needle is very closely tapered to the guidewire outer diameter so as to present negligible gap between the surfaces as is desirable for Seldinger access. At the distal end of the needle there is a magnetic needle actuator (6) that may be releasable from the needle.

The thin walled needle is flexible such that it is difficult to kink or crimp against the shaft of the guidewire under torsional mechanical loading. The guidewire and needle can become locked together in motion by the EMMT handle, thereby obviating potential guidewire shearing as a result of guidewire retraction during torsional loading of the exposed guidewire-needle interface within the tissues of the patient. Additionally, a beveled needle could be rotated 180 degrees from the puncture position, traditionally “bevel up” to aid entry, to a “bevel down” position. The bevel down position will protect the barb of the needle by bracing it along the inner curvature of the guidewire as it passes through the tissues. This sort of locking maneuver can be accomplished temporarily by one or two hands in the traditional approach of Seldinger access, but, is generally not possible to conceptualize the passage of a dilator (3) and sheath (4) over the needle and guidewire in the traditional hand operated procedure. The needle is typically removed completely by hand over the back of the protruding guidewire before placing the dilator and sheath over the wire and into the target tissue. This is generally not possible in an EMMT system without significant engineering complexity of completely removing the needle and then passing the dilator over the wire all under magnetic sterile enclosed actuation. The EMMT STA system can overcome this challenge through the “virtual locking” process whereby the needle is retracted into the dilator and “virtually” becomes a nested and locked part of the dilator through EMMT (Electromagnetic Motion and Tracking). Thus, the maneuvers of “virtual locking” and bevel up to bevel down rotation of the needle is a significant advantage for function of the device. In some ways, it is a disadvantage to consider that the first needle must remain in place over the central working wire. However, there are significant safety and waste containment benefits that are gained in constraining the key needle as well as guidewire within an EMMT cartridge for disposal. Indeed, it is the needle and guidewire that require strict regulatory handling protocols and pose the most risk to the operator and environment during and after procedural use.

The FIG. 4 device is operated in the following manner. First, the SUC (1, 2, 3, 4, 5, 6, 7, 8, 10,11) is mounted to the MCH handle (12 in FIG. 5), which contains and integrates a needle actuator (14) and guidewire actuator (13) The SUC is mounted with the needle and guidewire retracted within the dilator (3) and sheath (4) for protection and safe handling. The cartridge tubular housing (10) is long enough to facilitate complete retraction of the needle and guidewire into the cartridge housing (10) after the sheath is placed in order to protect the operator and environment from needlestick injury and contact/droplet contamination. The dilator (3) and sheath (4) may have a protective cover that is removed before use and could be used as a sleeve cover for the MCH (11). The front end cover is removed, for example, in the sterile field after mounting to the EMMT handle which has been sterilized, or covered to be sterile by a sleeve (11) which may be fused to the cartridge housing proximally. The operator grasps the entire system initially by the exposed sheath (4) with a sterile gloved hand. The operator directs the EMMT to advance the needle by, for example, 5 cm. At this point the operator may switch grasp to hold the shaft of the needle which has been exposed and may guide this into the vessel using standard ultrasound guidance. Alternatively, the exposed sheath (4) could be used as a temporary “handle”. After the needle tip has entered the vessel lumen, the machine may sense that there is now blood pool contact within the lumen of the needle, and, the guidewire is advanced and/or may be cued by the operator, or, could be completely automated to trigger if sensors indicate proper positioning and conditions of all elements. For example, in a more EMMT—manual hybrid scenario, the operator's first hand would grasp the EMMT system at the exposed sheath (4) in a pencil grip, then the operator would signal exposing the needle. Next the operator would advance the needle through the skin and into the vessel lumen under sterile probe ultrasound guideance. After entering the vessel lumen with the needle tip, the operator would use his/her thumb or index finger to push a button on the EMMT handle (5) which would advance and possibly simultaneously rotate the guidewire by mechanical and/or EMMT means. After a suitable length of guidewire is advanced into the vessel lumen unimpeded, the guidewire shaft will transition to a stiffer body, matched to the strength of the needle and suitable protecting the needle from inducing additional laceration, thereby disabling the cutting function needle.

Additional rotation of a beveled tip needle may be incorporated to allow additional protection of the barb of the needle along the concavity of an access guidewire (1). A flexible needle (2) may also be utilized. When the suitable length of wire is advanced, the needle (2) and guidewire (1) can be mechanically locked together in a fixed and static relative position to one another in order to be advanced as a unit under EMMT control safely, locked together for a short distance, for example 1-3 cm, into the vessel lumen in order to facilitate eventual dilator and overlying sheath/catheter passage required for achievement of Seldinger type access and modifications thereof. The specific facilitation of the process occurs through maintenance of a continuous, gapless and stepwise telescoping progression of diameters of the tools to the final step of implantation of the catheter or sheath implant, which is the procedural goal. The locking of needle to guidewire would occur on operator cue to the EMMT STA system handle, possibly a pushbutton with an indicator light. A skin incision is then made at and including the needle entry site, or the needle may have been passed through a previous skin incision made for this purpose initially. The guidewire and needle are now fused and the needle tip is positioned a short distance, about 1-3 cm (possibly 0.1 cm to 10 cm), into the vessel now facilitating passage of the dilator over the outside of the needle, parting the tissues of the vessel wall while closely tapered to the outer diameter of the needle. The dilator (3) and sheath (4) are then manually advanced by the operators first hand while the operator's second hand uses the EMMT MCH handle (12) to pin the central guidewire (1) and needle (2) from motion while the dilator (3) and sheath (4) are advanced relative to the MCH handle (12) and the internal needle (2) and guidewire (1). When the fluid blood pool is encountered within the vessel wall, the inherent gap between the outer diameter of the guidewire and the inner diameter of the dilator does not present any mechanical impediment or tissue damage risk after the 1-3 cm length of distal needle is traversed. Eventually, the dilator tip would pass into the vessel lumen and allow minimally unimpeded passage of a closely overlying thin walled, large diameter, sheath tube typically 4-6 F but up to 18-28 F inner diameter. Multiple nested dilator systems could be utilized with multiple associated sequential dilator actuators (7) in the position of (3). This may have mechanical advantages that an elongated tapered single dilator may lack. Note that F=diameter (mm)×3.

FIG. 5: Needle-Guidewire-Dilator-Sheath EMMT STA system, cross-section

FIG. 5 features additional EMMT integration of the dilator (3) and sheath (4), which were hand operated in concept 1, giving the operator additional and total MCH functionality and assistance for STA. This will require a slightly larger and possibly elastic front orifice cartridge diameter to accommodate the diameter of the dilator (3) and sheath (3) internally. It could require 2 distinct operator hand positions. The first operator hand position would be “needle exposed, guidewire retracted.” In this first position, the operator is grasping the MCH handgrasp, or, is grasping the bare exposed length of needle shaft in the usual 45 degree Seldinger approach, and, is thereby supporting the entire EMMT handle floating on bungee attachment via the needle shaft. The needle could also be uncoupled from the handle magnet for more free hand feel and then could be recoupled when the operator desires, as long as the internal length and surfaces of guidewire and unexposed needle are engineered to be sufficient to provide a stable, slidable mechanical coupling. For the second position, the operator would grasp the MCH handle and assume control of the guidewire (1), the needle (2), the sheath catheter/dilator (4,3) which may eventually become virtually locked to the needle (2). The final maneuver would be separation of the sheath-catheter (4) from the SUC (10) and magnetically coupled MCH (12) for implantation.

The single use cartridge (10) is mounted to the motion control handle (12) allowing up to 4 independent magnetic actuators which are physically located outside of the sterile field (13 guidewire actuator, 14 needle actuator, 15 dilator actuator(s), and 16 sheath/catheter actuator) to couple to matched actuators (5 guidewire actuator, 6 needle actuator, 7 dilator actuator, and 8 sheath/catheter actuator) which are located within the cartridge and within the sterile field. These elements are physically coupled through the sterile barrier cartridge housing by magnetic forces of attraction. The magnetic actuator linkages allows configurations of linkage between the handle and the internal components that may incorporate combinations of (a) magnetic linkage or decoupling, (b) coordinated computer controlled motion of the motors cued by the operator, (c) operator manipulation of the motion control handle hanging from its support stand, (d) optional operator hand manipulation and possible magnetic coupling and uncoupling of exposed internal components, and, (d) slidable extension or retraction of the SUC (10) outer tubular housing relative to the MCH (12) and patient's skin.

The operator advances the needle (2) into the vessel by hand under ultrasound guidance. When the needle tip is well centered within the vessel lumen, the operator signals or manipulates a joystick handle to advance/retract/rotate the internal guidewire via the EMMT MCH (12). The operator can have a direct tactile appreciation of the guidewire (1) by holding the MCH (12) relative to the freely floating exposed needle (2), which is decoupled from the MCH. If, as the guidewire is advanced, it encounters an obstruction to its advancement, there will be a counterforce consistent with the obstruction that will be translated to the MCH handle and thereby to the operator's MCH supporting hand, pushing this hand away from the free floating needle supporting hand. The handle may be additionally rotationally coupled relative to the internal guidewire, which may be designed with a curved tip, and the entire EMMT handle may be rotated manually within a rotatable mount, potentially giving the operator a steering capability using the rotatable “directional seeking” tip in situations where this may be useful. When a length of guidewire approximately 12 cm (for example, between 10 and 15 cm) has been advanced, the guidewire shaft has stiffened considerably, allowing the operator to grasp the shaft of the needle and push the handle forward against this to magnetically recouple the needle to the MCH for an again complete EMMT system. This configuration allows the operator to make a skin incision if this has not already been performed, and, then to signal the EMMT handle to advance a dilator to the tip of the needle and then beyond. At some point in the process, the needle (if it is beveled) may be triggered to rotate 180 degrees from the puncture “bevel up” position to a “bevel down” position to protect the barb of the needle from damaging tissues during motion. This entire process automated within the EMMT handle on operator cue and the weight of the entire process and the complex tissue dilation forces will be translated into the MCH and thereby to the operator's hand. The operator will feel a back pressure on the device handle, and, the operator will have to maintain forward pressure on the handle so as to not lose forward positioning of the needle tip between, for example, 1-3 cm beyond the endothelial inner vessel wall layer when giving cue to the system to, for example, advance the sheath over a locked needle and guidewire. The EMMT handle will provide a very gratifying tactile experience to the operator which will inspire inspire extreme operator confidence. Additionally, the end sheath/catheter implant (4) is maximally protected from ambient environment exposure prior to implantation, and, contaminated sharps medical waste, the needle (2) and guidewire (1) are securely contained before, during and after the procedure.

What is claimed:

1. A catheter or guidewire placement apparatus, comprising:
a sterile enclosed tube comprising: a needle, a guidewire, and a first ferromagnetic component coupled to the guidewire, and, optionally, a catheter;
one or more external magnets that are mounted to the exterior of the enclosed tube and coupled to one or more ferromagnetic components within the sterile tube;
and wherein the largest dimension of the enclosed tube is the length direction and wherein the one or more external magnets comprises: a first external magnet that is coupled to the first ferromagnetic component that is coupled to the guidewire and wherein the first external magnet is translatable in the direction of the length of the enclosed tube; and
a decoupling mechanism that allows the guidewire or needle to uncouple from the one or more external magnets.

2. The apparatus of claim 1 wherein the decoupling mechanism allows the needle to reversibly or irreversibly uncouple from the one or more external magnets.

3. The apparatus of claim 1 wherein the decoupling mechanism allows the guidewire to reversibly uncouple from the one or more external magnets.

4. The apparatus of claim 1 wherein the decoupling mechanism allows the guidewire to irreversibly uncouple from the one or more external magnets.

5. The apparatus of claim 1 wherein the decoupling mechanism creates a change in position of the magnetic actuator such that the central orifice becomes closed and impinges, crimps and locks upon the guidewire.

6. A catheter or guidewire placement apparatus, comprising:
a sterile enclosed tube comprising: a needle, a guidewire, and a first ferromagnetic component coupled to the guidewire, and, optionally, a catheter;
one or more external magnets that are mounted to the exterior of the enclosed tube and coupled to one or more ferromagnetic components within the sterile tube;
and wherein the largest dimension of the enclosed tube is the length direction and wherein the one or more external magnets comprises: a first external magnet that is coupled to the first ferromagnetic component that is coupled to the guidewire and wherein the first external magnet is translatable in the direction of the length of the enclosed tube; and
wherein the sterile enclosed tube is suspended from an overhead support by a bungee cord.

7. A catheter or guidewire placement apparatus, comprising:
a sterile enclosed tube comprising: a needle, a guidewire, a dilator, a sheath, and, optionally, a catheter;
the guidewire disposed within the needle;
wherein the sheath and dilator are cylindrical;
the dilator is slidable over the guidewire and the sheath slidable over the dilator;
and preferably where the sterile enclosed tube is suspended from an overhead support and preferably suspended from the support by a bungee cord.
